SEQUENCE LISTING (7 sequences)

SEQ ID NO: 1 - DNA, 20 bp, Artificial Sequence
  Synthetic DNA
ggttccaggtgatcaaccaa

SEQ ID NO: 2 - DNA, 22 bp, Artificial Sequence
  Synthetic DNA
gttccagttctaaggacgtctg

SEQ ID NO: 3 - DNA, 938 bp, Artificial Sequence
  Synthetic DNA
gaatgttgtgataaaaggtgatgctcacctctcccacacccttttatagtttagggattgtatttccaaggtttctagactgagagcccttttcatctttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccacgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctt

SEQ ID NO: 4 - DNA, 937 bp, Artificial Sequence
  Synthetic DNA
gaatgttgtgataaaaggtgatgctcacctctcccacacccttttatagtttagggattgtatttccaaggtttctagactgagagcccttttcatctttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccagaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctt

SEQ ID NO: 5 - DNA, 936 bp, Artificial Sequence
  Synthetic DNA
gaatgttgtgataaaaggtgatgctcacctctcccacacccttttatagtttagggattgtatttccaaggtttctagactgagagcccttttcatctttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctt

SEQ ID NO: 6 - DNA, 935 bp, Artificial Sequence
  Synthetic DNA
gaatgttgtgataaaaggtgatgctcacctctcccacacccttttatagtttagggattgtatttccaaggtttctagactgagagcccttttcatctttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggacccgaagtgttggatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctt

SEQ ID NO: 7 - DNA, 926 bp, Artificial Sequence
  Synthetic DNA
gaatgttgtgataaaaggtgatgctcacctctcccacacccttttatagtttagggattgtatttccaaggtttctagactgagagcccttttcatctttgctcattgacactctgtacccattaatcctccttattagctccccttcaatggacacatgggtagtcagggtgcaggtctcagaactgtccttcaggttccaggtgatcaaccaagtgccttgtctgtagtgtcaactcattgctgccccttcctagtaatccccataatttagctctccatttcatagtctttccttgggtgtgttaaaagtgaccatggtacactcagcacggatgaaatgaaacagtgtttagaaacgtcagtcttctcttttgtaatgccctgtagtctctctgtatgttatatgtcacattttgtaattaacagcttgctggtgaaaaggaccccatataagccagactgtaagtgaattactttttttgtcaatcatttaaccatctttaacctaaaagagttttatgtgaaatggcttataattgcttagagaatatttgtagagaggcacatttgccagtattagatttaaaagtgatgttttctttatctaaatgatgaattatgattctttttagttgttggatttgaaattccagacaagtttgttgtaggatatgcccttgactataatgaatacttcagggatttgaatgtaagtaattgcttctttttctcactcatttttcaaaacacgcataaaaatttaggaaagagaattgttttctccttccagcacctcataatttgaacagactgatggttcccattagtcacataaagctgtagtctagtacagacgtccttagaactggaacctggccaggctagggtgacacttcttgttggctgaaatagttgaacagctt

(19) United States
(12) Patent Application Publication
(10) Pub. No.: US 2020/0407776 A1
(43) Pub. Date: Dec. 31, 2020

(54) COMPOSITIONS AND METHODS FOR IMPROVED DETECTION OF GENOMIC EDITING EVENTS

(71) Applicant: Integrated DNA Technologies, Inc., Coralville, IA (US)

(72) Inventors: Christopher A Vakulskas, North Liberty, IA (US); Michael A Collingwood, North Liberty, IA (US); Mark A Behlke, Coralville, IA (US)

(21) Appl. No.: 16/912,132

(22) Filed: Jun. 25, 2020

Related U.S. Application Data
(60) Provisional application No. 62/866,806, filed on Jun. 26, 2019.

(51) Int. Cl. C12Q 1/686; C12Q 1/34

(52) U.S. Cl. CPC C12Q 1/686; G01N 2333/922; C12Q 2521/301; C12Q 1/34

(57) ABSTRACT

This invention pertains to compositions and methods for detecting single and/or multiple nucleotide mismatches on DNA fragments in vitro by enzymatic cleavage using a mixture of mismatch endonucleases. Additionally, this invention pertains to the ability to detect successful genome editing events by programmable nucleases, e.g., TALENS, RGENs, or ZFNs.

### CROSS REFERENCE TO RELATED APPLICATIONS

This application claims priority to U.S. Provisional Patent Application No. 62/866,806 filed on Jun. 26, 2019, the contents of which are incorporated by reference herein in their entirety.

### FIELD OF THE INVENTION

This invention pertains to the ability to detect single and/or multiple nucleotide mismatches on DNA fragments in vitro by enzymatic cleavage using a mixture of mismatch endonucleases. Additionally, this invention pertains to the ability to detect successful genome editing events by programmable nucleases, e.g., TALENS, RGENs, or ZFNs.

### INCORPORATION BY REFERENCE OF MATERIAL SUBMITTED ELECTRONICALLY

Incorporated by reference in its entirety herein is a computer-readable nucleotide/amino acid sequence listing submitted concurrently herewith and identified as follows: One 8,192 Byte ASCII (text) file named Seq_Listing.txt created on Jun. 25, 2020.

### BACKGROUND OF THE INVENTION

Targeted genomic editing has become a powerful tool and permits specific changes to be introduced into the genome of interest. Targeted genomic editing enables modifying genomes to correct defective genes, the introduction of new genes into the target genome or studying gene function. A number of systems may be employed for targeted genomic editing and include programmable nuclease systems. Programmable nuclease systems include systems, such as but not limited to, TALENS, ZFNs, or RNA-Guided Endonucleases (RGEN) like Cas9 or Cpf1.

Following introduction of a double-stranded break in the genomic DNA with a programmable nuclease, various host cell repair pathways heal the lesion via either non-homologous end joining (NHEJ), which typically introduces mutations or indels at the cut site that frequently lead to gene disruption through frameshift mutation) or via homology directed repair (HDR) if a suitable template nucleic acid is available. Prior methods involve the use of single endonucleases which suffer from a number of drawbacks. Single endonucleases are unable to cleave certain mismatch types, require highly purified DNA or exhibit non-specific DNA cleavage activity.

Some enzymes are very sensitive to buffer composition and contaminants present in reactions from PCR, so assays performed using these enzymes require purified nucleic acid samples, which increases cost, increases the time needed to perform the assay, and increases the yield needed for input PCR product, all undesired features.

Estimates of DNA editing may also be obtained using DNA sequencing methods, however these methods are costly and take days to weeks to perform.

The ability to accurately and quickly detect single and/or multiple genomic nucleotide mismatch in DNA fragments and unpurified nucleic acid samples is needed.

### BRIEF SUMMARY OF THE INVENTION

This invention pertains to the ability to detect single and/or multiple nucleotide mismatches on DNA fragments in vitro by enzymatic cleavage using a mixture of mismatch endonucleases. In some embodiments the mixture of mismatch endonucleases includes phage T7 endonuclease I and T4 endonuclease VII. This cleavage system discriminates DNA mismatches in both short (1 to 5) and long (6 or more) heteroduplexed double-stranded DNA (dsDNA) molecules. Though this invention is broadly applicable to the detection of mismatches present in any DNA heteroduplex sample, it offers improvement in the ability to detect successful genome editing events by targeted genomic editing nucleases, e.g., TALENS, RGENs, or ZFNs.

Programmable nuclease systems allow for cleavage of complex DNA at precise positions in live cells. Following introduction of a double-stranded break in the genomic DNA with TALENS, RGENs, or ZFNs nucleases, various host cell repair pathways heal the lesion via either non-homologous end joining (NHEJ), which typically introduces mutations or indels at the cut site that frequently lead to gene disruption through frameshift mutation) or via homology directed repair (HDR) if a suitable template nucleic acid is available.

Alteration in DNA sequence can be detected following NHEJ repair using a mismatch endonuclease cleavage assay. In this assay format, a PCR amplicon is first made from genomic DNA isolated from treated cells that spans the expected targeting nuclease cut site. Following completion of PCR, the amplicons are denatured and allowed to anneal, leading to the formation of homoduplex wild-type amplicons or heteroduplex amplicons between wild-type and mutant molecules or between different mutant molecules. The mixture of homoduplex and heteroduplex DNA strands are treated with a mismatch endonuclease (typically Cel-I, Surveyor, or T7 endonuclease I, T7E1), which cleaves the DNA where mismatch bubbles are present. Finally, a visualization method is employed to detect the cleavage event, such as, but not limited to, agarose gel electrophoresis or capillary electrophoresis (CE). Using CE, the relative amounts of cleaved (mutated) vs. uncleaved (homoduplex WT or homoduplex mutant) can be accurately measured.

Typically, targeted genomic editing experiments are carried out on large pools of immortalized animal or plant cells, and genomic DNA is subsequently extracted and purified as a mixture of both edited and unedited cells. The frequency by which targeted genomic editing facilitates successful cleavage depends on a number of variables including (but not limited to) cell type, target site sequence, and context of the flanking genomic DNA. Determining the percentage of both edited and unedited cells for targeted genomic editing cleavage is crucial to understand the relative success or failure for a given gene editing experiment

Since host-dependent repair enzymes facilitate multiple overlapping and poorly understood repair mechanisms, it is at present impossible to predict the repaired sequence that results from a targeted genomic editing dependent cleavage event. There are often multiple different types of repair outcomes for a given cleavage site that vary in frequency. DNA repair of a cleavage lesions can result in either short (1 to 5) or long (6 or more) base pair additions or deletions. There is currently no proven method to enzymatically digest unpurified DNA directly out of PCR that reliably targets both long and short mismatches.

The proposed method and compositions involves the use of a blend of two endonucleases. In some embodiments a mixture of T7EI and T4 Endonuclease VII (T4E7) is used to more efficiently cleave heteroduplexed DNA mismatches than can be resolved using a single enzyme. The present invention employs a mixture of endonucleases. In some embodiments the mixture of endonucleases comprises T7E1 with T4E7. The mixture of T7E1 and T4E7 perform better than either enzyme alone. The ability of the two enzymes to complement each other in function. Without being bound to any singular theory it is postulated that the T7EI in the mixture serves to cleave the majority of heteroduplexes containing 2 or more adjacent mismatches while the T4E7 serves to cleave single nucleotide mismatches. The basic procedure and premise is detailed in FIG. 1. Both enzymes are compatible in a simple nuclease cleavage reaction buffer (heteroduplex or HD Buffer) containing 10 mM Tris-HCl (pH 7.9), 50 mM NaCl, 10 mM MgCl2, and 1 mM DTT, and are highly active at 37 degrees Celsius. Importantly, the enzyme combination performs well on unpurified PCR heteroduplex products and works in PCR reaction buffer.

### BRIEF DESCRIPTION OF THE FIGURES

FIG. 1 is a diagram showing mismatch cleavage capabilities of the T7E1 and T4E7 endonucleases in isolation. The figure shows the cleavage of 1, 3 or 12 base pair insertions.

FIG. 2 shows the blend of T7E1 and T4E7 endonucleases and the capability of blend to detect mismatches of various sizes.

FIG. 3 shows a blend of T7E1 and T4E7 endonucleases and the capability to detect mismatches at CRISPR edited genomic sites. For each grouping of bars at each target site the left hand bar shows T7(1:10) vs the right hand bars that shows T7(1:10)+T4 (1:3).

### DETAILED DESCRIPTION OF THE INVENTION

The methods and compositions of the invention described herein provide enzyme compositions and methods for detecting single and/or multiple nucleotide mismatches introduced by genomic editing events with programmable nucleases. These and other advantages of the invention, as well as additional inventive features, will be apparent from the description of the invention provided herein.

This invention pertains to the ability to detect single and/or multiple nucleotide mismatches on DNA fragments in vitro by enzymatic cleavage using a mixture of mismatch endonucleases. In some embodiments the mixture of mismatch endonucleases includes phage T7 endonuclease I and T4 endonuclease VII. This cleavage system discriminates DNA mismatches in both short (1 to 5) and long (6 or more) heteroduplexed double-stranded DNA (dsDNA) molecules. Though this invention is broadly applicable to the detection of mismatches present in any DNA heteroduplex sample, it offers improvement in the ability to detect successful genome editing events by targeted genomic editing nucleases, e.g., TALENS, RGENs, or ZFNs.

The invention is targeted to mismatch discrimination detection for CRISPR/Cas9 genome editing but is broadly applicable to any use of this enzyme blend to cleave any DNA source containing mismatched nucleotides, such as assays intended to detect naturally occurring mutations as well as experimentally induced mutations.

In one embodiment the invention is targeted to mismatch discrimination detection for programmable nuclease genome editing. In another embodiment the invention is targeted to mismatch discrimination for TALENs, ZFNs, or RGENs. In an additional embodiment the invention is targeted to mismatch discrimination detection for RGEN genome editing. In a further embodiment the invention is targeted to mismatch discrimination detection for Cas9 or Cpf1 genome editing. In a further embodiment the invention is applicable to the use of this enzyme blend to cleave any DNA source containing mismatched nucleotides, such as assays intended to detect naturally occurring mutations as well as experimentally induced mutations.

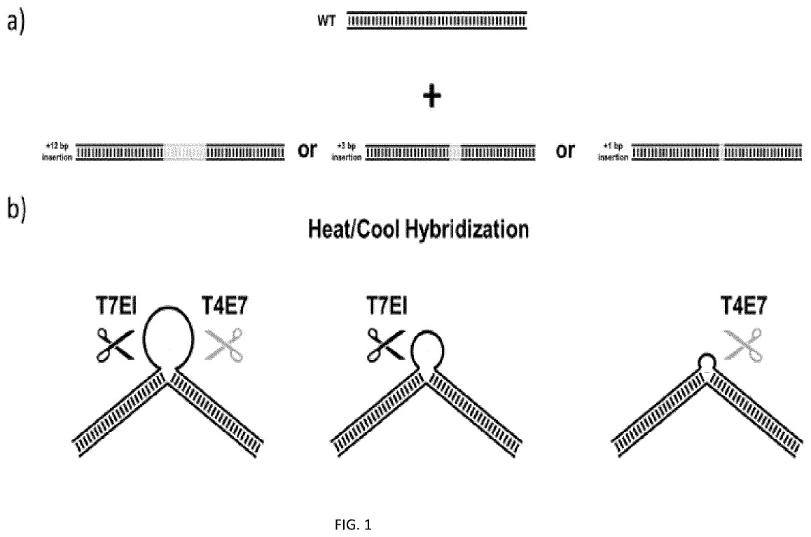

In one embodiment a double strand break (DSB) is introduced with a programmable nuclease system. The DSB is repaired through NHEJ or HDR events. Following NHEJ or HDR repair the repaired genome is optionally PCR amplified with primers spanning the predicted cut sites. The genomic editing events are then detected with a combination of mismatch endonucleases. In an additional embodiment the proposed method and compositions involve the use of a blend of at least two endonucleases. In one embodiment a mixture of T7EI and T4 Endonuclease VII (T4E7) is used to more efficiently cleave heteroduplexed DNA mismatches than can be resolved using a single enzyme. The mixture of T7E1 and T4E7 are able to cleave more types or repair fragments than either enzyme alone. The ability of the two enzymes to complement each other in function. Without being bound to any singular theory it is postulated that the T7EI in the mixture serves to cleave the majority of heteroduplexes containing 2 or more adjacent mismatches while the T4E7 serves to cleave single nucleotide mismatches. The basic procedure and premise is detailed in FIG. 1.

In FIG. 1, part a) shows a 1:1 mixture of 1 KB WT dsDNA and an otherwise identical 1 KB dsDNA strand that has a centered insertion/deletion of 12, 3, or 1 base(s) is prepared for hybridization. In part b) the samples are denatured at 95 degrees Celsius and heteroduplexes are formed by repeated heating and cooling to generate various mismatch dsDNA fragments. In isolation, T7E1 cleaves heteroduplexes with long and short mismatch bubbles (2-12) bases efficiently, but does not cleave single base mismatch bubbles. T4E7 cleaves large mismatch bubbles efficiently (12 bases), does not cleave short mismatch bubbles (2-3 bases), and is capable of cleaving single base mismatch bubbles with low efficiency.

Both enzymes are compatible in a simple nuclease cleavage reaction buffer (heteroduplex or HD Buffer) containing 10 mM Tris-HCl (pH 7.9), 50 mM NaCl, 10 mM MgCl2, and 1 mM DTT, and are highly active at 37 degrees Celsius.

In another embodiment the enzyme combination performs well on unpurified PCR heteroduplex products and works in PCR reaction buffer.

### Example 1

Utilization of the T4/T7 Nuclease Blend to Cleave Heteroduplexes Containing 1, 2, 3, and 12 Nucleotide Mismatches.

The following example demonstrates the ability of the method of the invention to efficiently cleave mismatched heteroduplexes PCR products more efficiently than traditional approaches using single enzyme protocols. PCR assays were designed to amplify a 1 kb fragment of the Human HPRT1 gene. HPRT gBlocks Gene Fragments were designed to contain 0(WT), 1, 2, 3, or 12 base deletions positioned at around base 300 of these amplicons, such that mismatch heteroduplex cleavage assays would yield fragments of ˜300 and ˜700 bp, sizes that are readily distinguished. The HPRT gBlocks gene fragments were amplified using the HPRT Forward primer and HPRT Rev Primer. Primers and DNA fragments are shown in Table 1 (SEQ IDs #1-7). These synthetic gBlocks gene fragments represent hypothetical repair fragments following genomic editing events using programmable nucleases.

TABLE 1
Sequence of oligonucleotides and gBlocks used to make heteroduplexes.

| | | SEQ ID |
|---|---|---|
| Name | Sequence | NO: |
| HPRT | GGTTCCAGGTGATCAACCAA | SEQ ID |
| Forward | | NO: 1 |
| primer | | |
| HPRT Rev | GTTCCAGTTCTAAGGACGTCTG | SEQ ID |
| primer | | NO: 2 |
| HPRT WT | GAATGTTGTGATAAAAGGTGATGCTCACCTCTCCCACACCCTTTTATAGTTTAGGGA | SEQ ID |
| gBlock | TTGTATTTCCAAGGTTTCTAGACTGAGAGCCCTTTTCATCTTTGCTCATTGACACTC | NO: 3 |
| | TGTACCCATTAATCCTCCTTATTAGCTCCCCTTCAATGGACACATGGGTAGTCAGGG | |
| | TGCAGGTCTCAGAACTGTCCTTCAGGTTCCAGGTGATCAACCAAGTGCCTTGTCTGT | |
| | AGTGTCAACTCATTGCTGCCCCTTCCTAGTAATCCCCATAATTTAGCTCTCCATTTC | |
| | ATAGTCTTTCCTTGGGTGTGTTAAAAGTGACCATGGTACACTCAGCACGGATGAAAT | |
| | GAAACAGTGTTTAGAAACGTCAGTCTTCTCTTTTGTAATGCCCTGTAGTCTCTCTGT | |
| | ATGTTATATGTCACATTTTGTAATTAACAGCTTGCTGGTGAAAAGGACCCCACGAAG | |
| | TGTTGGATATAAGCCAGACTGTAAGTGAATTACTTTTTTTGTCAATCATTTAACCAT | |
| | CTTTAACCTAAAAGAGTTTTATGTGAAATGGCTTATAATTGCTTAGAGAATATTTGT | |
| | AGAGAGGCACATTTGCCAGTATTAGATTTAAAAGTGATGTTTTCTTTATCTAAATGA | |
| | TGAATTATGATTCTTTTTAGTTGTTGGATTTGAAATTCCAGACAAGTTTGTTGTAGG | |
| | ATATGCCCTTGACTATAATGAATACTTCAGGGATTTGAATGTAAGTAATTGCTTCTT | |
| | TTTCTCACTCATTTTTCAAAACACGCATAAAAATTTAGGAAAGAGAATTGTTTTCTC | |
| | CTTCCAGCACCTCATAATTTGAACAGACTGATGGTTCCCATTAGTCACATAAAGCTG | |
| | TAGTCTAGTACAGACGTCCTTAGAACTGGAACCTGGCCAGGCTAGGGTGACACTTCT | |
| | TGTTGGCTGAAATAGTTGAACAGCTT | |
| HPRT | GAATGTTGTGATAAAAGGTGATGCTCACCTCTCCCACACCCTTTTATAGTTTAGGGA | SEQ ID |
| 1DEL | TTGTATTTCCAAGGTTTCTAGACTGAGAGCCCTTTTCATCTTTGCTCATTGACACTC | NO: 4 |
| gBlock | TGTACCCATTAATCCTCCTTATTAGCTCCCCTTCAATGGACACATGGGTAGTCAGGG | |
| | TGCAGGTCTCAGAACTGTCCTTCAGGTTCCAGGTGATCAACCAAGTGCCTTGTCTGT | |
| | AGTGTCAACTCATTGCTGCCCCTTCCTAGTAATCCCCATAATTTAGCTCTCCATTTC | |
| | ATAGTCTTTCCTTGGGTGTGTTAAAAGTGACCATGGTACACTCAGCACGGATGAAAT | |
| | GAAACAGTGTTTAGAAACGTCAGTCTTCTCTTTTGTAATGCCCTGTAGTCTCTCTGT | |
| | ATGTTATATGTCACATTTTGTAATTAACAGCTTGCTGGTGAAAAGGACCCCAGAAGT | |
| | TGTTGGATAAAGCCAGACTGTAAGTGAATTACTTTTTTTGTCAATCATTTAACCATC | |
| | TTTAACCTAAAAGAGTTTTATGTGAAATGGCTTATAATTGCTTAGAGAATATTTGTA | |
| | GAGAGGCACATTTGCCAGTATTAGATTTAAAAGTGATGTTTTCTTTATCTAAATGAT | |
| | GAATTATGATTCTTTTTAGTTGTTGGATTTGAAATTCCAGACAAGTTTGTTGTAGGA | |
| | TATGCCCTTGACTATAATGAATACTTCAGGGATTTGAATGTAAGTAATTGCTTCTTT | |
| | TTCTCACTCATTTTTCAAAACACGCATAAAAATTTAGGAAAGAGAATTGTTTTCTCC | |
| | TTCCAGCACCTCATAATTTGAACAGACTGATGGTTCCCATTAGTCACATAAAGCTGT | |
| | AGTCTAGTACAGACGTCCTTAGAACTGGAACCTGGCCAGGCTAGGGTGACACTTCTT | |
| | GTTGGCTGAAATAGTTGAACAGCTT | |
| HPRT | GAATGTTGTGATAAAAGGTGATGCTCACCTCTCCCACACCCTTTTATAGTTTAGGGAT | SEQ TD |
| 2DEL | TGTATTTCCAAGGTTTCTAGACTGAGAGCCCTTTTCATCTTTGCTCATTGACACTCTG | NO: 5 |
| gBlock | TACCCATTAATCCTCCTTATTAGCTCCCCTTCAATGGACACATGGGTAGTCAGGGTGC | |
| | AGGTCTCAGAACTGTCCTTCAGGTTCCAGGTGATCAACCAAGTGCCTTGTCTGTAGTG | |
| | TCAACTCATTGCTGCCCCTTCCTAGTAATCCCCATAATTTAGCTCTCCATTTCATAGT | |
| | CTTTCCTTGGGTGTGTTAAAAGTGACCATGGTACACTCAGCACGGATGAAATGAAACA | |
| | GTGTTTAGAAACGTCAGTCTTCTCTTTTGTAATGCCCTGTAGTCTCTCTGTATGTTAT | |
| | ATGTCACATTTTGTAATTAACAGCTTGCTGGTGAAAAGGACCCCGAAGTGTTGGATAT | |
| | AAGCCAGACTGTAAGTGAATTACTTTTTTTGTCAATCATTTAACCATCTTTAACCTAA | |
| | AAGAGTTTTATGTGAAATGGCTTATAATTGCTTAGAGAATATTTGTAGAGAGGCACAT | |
| | TTGCCAGTATTAGATTTAAAAGTGATGTTTTCTTTATCTAAATGATGAATTATGATTC | |
| | TTTTTAGTTGTTGGATTTGAAATTCCAGACAAGTTTGTTGTAGGATATGCCCTTGACT | |
| | ATAATGAATACTTCAGGGATTTGAATGTAAGTAATTGCTTCTTTTTCTCACTCATTTT | |
| | TCAAAACACGCATAAAAATTTAGGAAAGAGAATTGTTTTCTCCTTCCAGCACCTCATA | |
| | ATTTGAACAGACTGATGGTTCCCATTAGTCACATAAAGCTGTAGTCTAGTACAGACGT | |
| | CCTTAGAACTGGAACCTGGCCAGGCTAGGGTGACACTTCTTGTTGGCTGAAATAGTTG | |
| | AACAGCTT | |
| HPRT | GAATGTTGTGATAAAAGGTGATGCTCACCTCTCCCACACCCTTTTATAGTTTAGGGAT | SEQ ID |
| 3DEL | TGTATTTCCAAGGTTTCTAGACTGAGAGCCCTTTTCATCTTTGCTCATTGACACTCTG | NO: 6 |
| gBlock | TACCCATTAATCCTCCTTATTAGCTCCCCTTCAATGGACACATGGGTAGTCAGGGTGC | |
| | AGGTCTCAGAACTGTCCTTCAGGTTCCAGGTGATCAACCAAGTGCCTTGTCTGTAGTG | |
| | TCAACTCATTGCTGCCCCTTCCTAGTAATCCCCATAATTTAGCTCTCCATTTCATAGT | |
| | CTTTCCTTGGGTGTGTTAAAAGTGACCATGGTACACTCAGCACGGATGAAATGAAACA | |
| | GTGTTTAGAAACGTCAGTCTTCTCTTTTGTAATGCCCTGTAGTCTCTCTGTATGTTAT | |
| | ATGTCACATTTTGTAATTAACAGCTTGCTGGTGAAAAGGACCCGAAGTGTTGGATATA | |
| | AGCCAGACTGTAAGTGAATTACTTTTTTTGTCAATCATTTAACCATCTTTAACCTAAA | |
| | AGAGTTTTATGTGAAATGGCTTATAATTGCTTAGAGAATATTTGTAGAGAGGCACATT | |
| | TGCCAGTATTAGATTTAAAAGTGATGTTTTCTTTATCTAAATGATGAATTATGATTCT | |
| | TTTTAGTTGTTGGATTTGAAATTCCAGACAAGTTTGTTGTAGGATATGCCCTTGACTA | |
| | TAATGAATACTTCAGGGATTTGAATGTAAGTAATTGCTTCTTTTTCTCACTCATTTTT | |
| | CAAAACACGCATAAAAATTTAGGAAAGAGAATTGTTTTCTCCTTCCAGCACCTCATAA | |
| | TTTGAACAGACTGATGGTTCCCATTAGTCACATAAAGCTGTAGTCTAGTACAGACGTC | |
| | CTTAGAACTGGAACCTGGCCAGGCTAGGGTGACACTTCTTGTTGGCTGAAATAGTTGA | |
| | ACAGCTT | |
| HPRT | GAATGTTGTGATAAAAGGTGATGCTCACCTCTCCCACACCCTTTTATAGTTTAGGGAT | SEQ ID |
| 12DEL | TGTATTTCCAAGGTTTCTAGACTGAGAGCCCTTTTCATCTTTGCTCATTGACACTCTG | NO: 7 |
| gBlock | TACCCATTAATCCTCCTTATTAGCTCCCCTTCAATGGACACATGGGTAGTCAGGGTGC | |
| | AGGTCTCAGAACTGTCCTTCAGGTTCCAGGTGATCAACCAAGTGCCTTGTCTGTAGTG | |
| | TCAACTCATTGCTGCCCCTTCCTAGTAATCCCCATAATTTAGCTCTCCATTTCATAGT | |
| | CTTTCCTTGGGTGTGTTAAAAGTGACCATGGTACACTCAGCACGGATGAAATGAAACA | |
| | GTGTTTAGAAACGTCAGTCTTCTCTTTTGTAATGCCCTGTAGTCTCTCTGTATGTTAT | |
| | ATGTCACATTTTGTAATTAACAGCTTGCTGGTGAAAAGGACCCCAxxxxxxxxxxxTA | |
| | TAAGCCAGACTGTAAGTGAATTACTTTTTTTGTCAATCATTTAACCATCTTTAACCTA | |
| | AAAGAGTTTTATGTGAAATGGCTTATAATTGCTTAGAGAATATTTGTAGAGAGGCACA | |
| | TTTGCCAGTATTAGATTTAAAAGTGATGTTTTCTTTATCTAAATGATGAATTATGATT | |
| | CTTTTTAGTTGTTGGATTTGAAATTCCAGACAAGTTTGTTGTAGGATATGCCCTTGAC | |
| | TATAATGAATACTTCAGGGATTTGAATGTAAGTAATTGCTTCTTTTTCTCACTCATTT | |
| | TTCAAAACACGCATAAAAATTTAGGAAAGAGAATTGTTTTCTCCTTCCAGCACCTCAT | |
| | AATTTGAACAGACTGATGGTTCCCATTAGTCACATAAAGCTGTAGTCTAGTACAGACG | |
| | TCCTTAGAACTGGAACCTGGCCAGGCTAGGGTGACACTTCTTGTTGGCTGAAATAGTT | |
| | GAACAGCTT | |

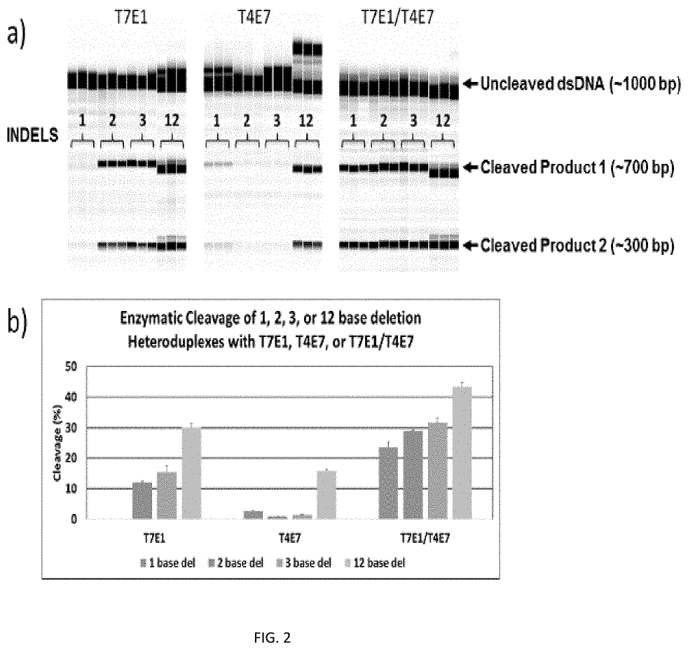

Following PCR equimolar mixtures (3 pmole each) of each deletion mutant and WT sequence were made in 1×HD buffer and the DNA was denatured and hybridized by heating followed by a step down cooling protocol in a thermocycler to generate heteroduplex fragments. The WT sequence and each deletion mutant were denatured and slow cooled to generate various heteroduplex fragments with the following cycling parameters: 95° C.^{10:00} cooled to 85° C. over 1 min, 85° C.^{1:00} cooled to 75° C. over 1 min, 75° C.^{1:00} cooled to 65° C. over 1 min, 65° C.^{1:00} cooled to 55° C. over 1 min, 55° C.^{1:00} cooled to 45° C. over 1 min, 45° C.^{1:00} cooled to 35° C. over 1 min, 35° C.^{1:00} cooled to 2° C. 5 over 1 min, 25° C.^{1:00}. These 50/50 mixtures of homoduplex/heteroduplex DNAs were then cleaved using either 0.38 pmol T7EI or 0.19 pmol T4E7 alone for 1 hr at 37° C. or a combination of both enzymes (0.38 pmol T7EI and 0.19 pmol T4E7 mix together) for 1 hr at 37° C. The reactions (20 μl) were then diluted with 0.15 ml 0.1×TE (10 mM Tris-HCl pH 7.5, 0.1 mM EDTA) and cleaved products were analyzed with a CE instrument, the Fragment Analyzer (Advanced Analytical). Experiments were performed in triplicate and results are shown in FIG. 2.

FIG. 2 a) shows the results following treatment with the single enzymes or the T4/T7 endonuclease mix on a Fragment Analyzer electropherogram. The Fragment Analyzer electropherogram in FIG. 2 a) shows the cleavage profiles with T7E1 alone, T4E7 alone, or mixture of both enzymes. Cut substrates include heteroduplexes with the indicated number of mismatched nucleotides (1, 2, 3, or 12). FIG. 2 b) shows quantitative data from FIG. 2a (top) as a function of cleavage percentage. Error bars represent the standard errors of the means.

Results show that as individual enzymes, T7EI alone recognized 2, 3, and 12 base mismatches better than the T4E7 enzyme alone. The T7EI enzyme alone cleaved all heteroduplexes except those having a single indel, which remained uncleaved. T4E7 enzyme alone weakly cleaved the single indel species, and also cleaved fragments having 2 or 3 base indels at a lower rate than the T7EI enzyme alone. Both enzymes alone cleaved the heteroduplex with a large 12 base indel. However, the T7EI+T4E7 cocktail mixture cleaved all of the heteroduplex fragment species. It was also noted that the combination of enzymes was able to cleave all fragment species more efficiently than either enzyme alone.

### Example 2

The following example demonstrates the ability the combination of T7EI and T4E7 to more efficiently cleave mismatched heteroduplex PCR amplified genomic edited products as compared to T7EI alone.

Following genomic editing with a CRISPR genomic editing system target sites were PCR amplified. Following PCR amplification, the target sites were enzymatically treated with either T7E1 enzyme alone or a mixture of T7E1 enzyme and T4E7 enzyme. Following enzymatic the target sample were run on a capillary electrophoresis and the cleavage percentage was determined.

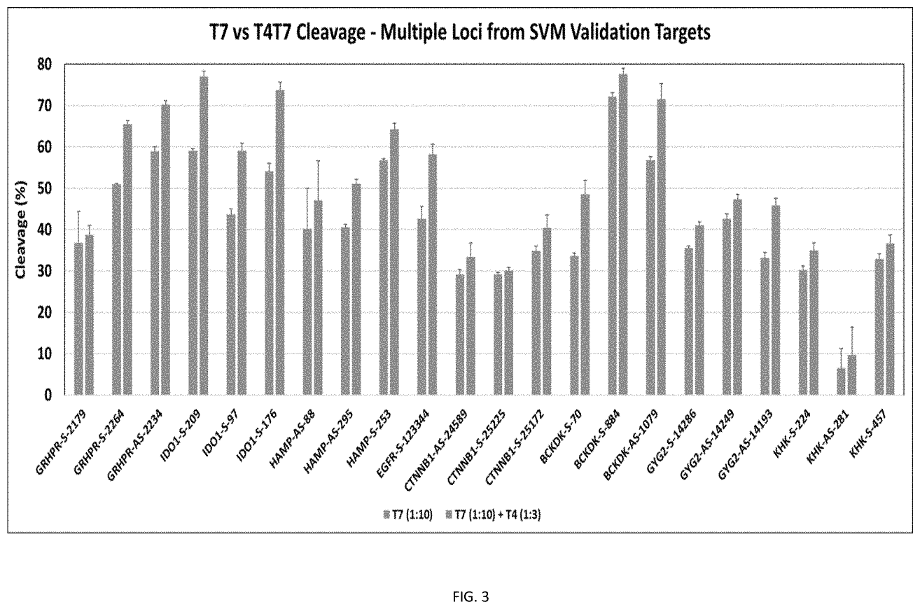

FIG. 3 shows the cleavage percentage of multiple loci following enzymatic treatment with T7E1 alone or a mixture of T7E1 enzyme and T4E7 enzyme. As the figure shows the mixture of T7E1 and T4E7 is able to more accurately determine the true cleavage percentage.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising”, “having”, “including” and “containing” are to be construed as open-ended terms (i.e., meaning “including, but no limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EMBODIMENTS

A1. A method for detecting genomic editing events comprising:
a) obtaining an edited genomic sequences;
b) PCR amplifying the edited genomic sequences around the expected edited region to generate a plurality of PCR amplicons;
c) reacting the plurality of PCR with a mixture of mismatch endonucleases to generate a plurality of cleaved fragments;
d) detecting the cleaved fragments indicating a genomic editing event.

A2. The method of claim A1 wherein the edited genomic sequence edited with a programmable nuclease.

A3. The method of claim A2 wherein the programmable nuclease is a TALEN, a RGEN, or a ZFN.

A4. The method of claim A3 wherein the RGEN is a Cas9 enzyme or a Cpf1 enzyme.

A5. The method of claim A1 wherein the mixture of mismatch endonucleases comprises T7EI and T4E7.

A6. An enzyme composition for detecting edited genomic DNA comprising a mixture of mismatch endonucleases.

A7. The composition of claim A6 wherein the mixture of mismatch endonucleases consists of T7E1 and T4E7.

A8. The composition of claim A7 wherein the mixture of mismatch endonucleases consists of 0.38 pmol T7EI and 0.19 pmol T4E7.

### REFERENCES

U.S. Pat. No. 5,698,400A DETECTION OF MUTATION BY RESOLVASE CLEAVAGE
Mashal R. D., Koontz J., and Sklar J. Detection of mutations by cleavage of DNA heteroduplexes with bacteriophage resolvases. Nat Genet., 1995 9:177-83.
Babon J. J., McKenzie M., and Cotton R. G. The use of resolvases T4 endonuclease VII and T7 endonuclease I in mutation detection. Methods Mol Biol., 2000, 152:187-99.
Mean, R. J., Pierides, A., Deltas, C. C., and Koptides, M. Modification of the enzyme mismatch cleavage method using T7 endonuclease 1 and silver staining. BioTechniques, 2004, 36:758-760.
Vouillot, L., Thelie, A., and Pollet, N. Comparison of T7E1 and Surveyor mismatch cleavage assays to detect mutations triggered by engineered nucleases. Genes, Genomes, Genetics, 2015, 5:407-415.

What is claimed is:

1. A method for detecting genomic editing events comprising:
a) obtaining an edited genomic sequences;
b) PCR amplifying the edited genomic sequences around the expected edited region to generate a plurality of PCR amplicons;
c) reacting the plurality of PCR amplicons with a mixture of mismatch endonucleases to generate a plurality of cleaved fragments;
d) detecting the cleaved fragments indicating a genomic editing event.

2. The method of claim 1 wherein the edited genomic sequence is edited with a programmable nuclease.

3. The method of claim 2 wherein the programmable nuclease is a TALEN, a RGEN, or a ZFN.

4. The method of claim 3 wherein the RGEN is a Cas9 enzyme or a Cpf1 enzyme.

5. The method of claim 1 wherein the mixture of mismatch endonucleases comprises T7EI and T4E7.

6. An enzyme composition for detecting edited genomic DNA comprising a mixture of mismatch endonucleases.

7. The composition of claim 6 wherein the mixture of mismatch endonucleases comprises T7E1 and T4E7.

8. The composition of claim 7 wherein the mixture of mismatch endonucleases comprises 0.38 pmol T7EI and 0.19 pmol T4E7.
